(19) United States
(12) Patent Application Publication
(10) Pub. No.: US 2024/0252437 A1
(43) Pub. Date: Aug. 1, 2024

(54) Methods For and Products From Encapsulation of Drugs in Nanoparticles in a Microgravity Environment

(71) Applicant: Aphios Corporation, Arlington, MA (US)

(72) Inventor: Trevor Percival Castor, Arlington, MA (US)

(73) Assignee: Aphios Corporation, Arlington, MA (US)

(21) Appl. No.: 18/634,819

(22) Filed: Apr. 12, 2024

Related U.S. Application Data
(63) Continuation of application No. 17/221,831, filed on Apr. 4, 2021.

(51) Int. Cl. A61K 9/127; A61K 31/35; A61K 47/24

(52) U.S. Cl. CPC A61K 9/1277; A61K 31/35; A61K 47/24

(57) ABSTRACT

A method is disclosed for producing targeted nanoencapsulated therapeutics in a microgravity environment using supercritical, critical and near-critical fluids with and without polar cosolvents. Using the disclosed technology, nanosomes for delivering Bryostatin-1 and other Bryoids are produced in microgravity. The resulting nanosomes are smaller, more uniform, with a higher surface area to volume than those produced in gravity-based environments and have an average diameter between 0.001 to 20.000 nanometer. The resultant therapeutics may be used for treating chronic diseases such as cancer, HIV, and Alzheimer's disease.

### GOVERNMENT SUPPORT

Research leading to this invention was in part funded with Grant No. GA-2018-275 from the Center for the Advancement of Science in Space, Inc., (“CASIS”), Melbourne, FL, which manages the International Space Station (“ISS”) National Laboratory (“NL”) in accordance with NASA Cooperative Agreement No. NNH11CD70A.

### FIELD OF THE INVENTION

This invention relates to methods for and products from precision manufacturing targeted nanoencapsulated drugs in a low gravity or microgravity environment utilizing green, environment friendly SuperFluids™.

### BACKGROUND OF THE INVENTION

Based on these methods, nanoparticles formulation of molecules such as, but not limited to Bryostatin-1 and other Bryoids, manufactured and lyophilized in a gravity-based environment, are smaller and more uniform with a higher surface area to volume ratio, approaching ‘picometer dimensions’ in a microgravity environment. Once frozen in Space, these enhanced nanoparticles approaching ‘picometer dimensions’ also find utility on Earth to treat ‘orphan’ and chronic diseases such as cancer, HIV and Alzheimer's disease. The methods feature SuperFluids™ which are supercritical, critical and near-critical fluids with and without polar cosolvents.

### SUMMARY OF THE INVENTION

Microgravity, microgravity environment, or microgravity conditions are defined as a very low gravity force of less than 1×10⁻³×g. The units for microgravity are expressed as a multiplier of normal terrestrial gravity (g) as measured on the surface of the earth. This definition of microgravity as less than 1×10⁻³×g is supported by the National Aeronautics and Space Administration of the United States. Microgravity may be achieved in certain trajectories of rockets. Microgravity may be achieved in reduced-gravity aircraft flying in a parabolic trajectory. A reduced gravity aircraft is a type of fixed-wing aircraft that provides brief near-weightless environments for training astronauts, conducting research, and making gravity-free movie shots. Even longer periods of microgravity may be obtained in orbital or suborbital spacecraft such as the NASA space shuttle. Relatively indefinite periods of microgravity may be obtained in permanent or semi-permanent orbital space stations such as the International Space Station (ISS) and in orbital satellites, such as geosynchronous orbital satellites. Microgravity may be obtained in interplanetary space probes and spacecraft.

Targeted nanoparticle delivery systems are precision manufactured using environmental-friendly SuperFluids™ technologies for use in ‘orphan’ and chronic diseases, like cancer, HIV and Alzheimer' disease, in the microgravity environment of the International Space Station (ISS). These targeted nanoparticles can be used on earth to treat a variety of different types of diseases. The novel precision method for creating these targeted nanoparticles can reduce both costs and environmental impacts of drug development. The production of these precision targeted nanoparticles in microgravity environments such as space expands opportunities for precision targeted medicine manufacturing on the ISS, with precision targeted medicine manufacturing becoming a highly valuable commerce on Earth. Several manufacturing and testing issues particularly with nanoencapsulation encountered in normal gravity currently limit yields, quality and validation of these medicine-delivery strategies limiting their translation into the clinic. The manufacturing of precision targeted nanoparticles therapeutics in space is a significant innovation in the production of therapeutics that accelerates the treatment of disease on Earth.

In one aspect, the size reduction of the encapsulating nanoparticles gives major advantages in the creation of rare therapeutics. The size reduction of nanoparticles created in microgravity vastly increases efficiency for uptake and delivery, reducing the required dose per treatment and cost per dose, and enormously increasing production value. Precision microgravity-manufactured targeted nanoparticles, therefore, have an extraordinarily high potential to treat and potentially cure several orphan and chronic diseases.

Alzheimer's Disease—A Highly Unmet Medical Need. Alzheimer's disease (AD) is the seventh leading cause of death in the US and is among the highest in the industrial world. AD is a neurological disorder that affects >55 million people worldwide. According to the Alzheimer's Disease Association more than 6.7 million Americans older than 65 will suffer from AD in 2023 with black Americans more than twice as likely to succumb to the disease. More than 55 million people worldwide were living with dementia and AD in 2020. Without a cure and/or preventative treatments, AD prevalence is projected to increase with the increased aged populations in the United States and the world. Epidemiologically, AD increases with the demographics of aging populations in US, Europe and Japan. Experts estimate that by 2050, >81 million people around the world and more than 14 million Americans will be afflicted with AD comprising an enormous clinical market. The Alzheimer's Association estimates that AD cost the United States $345 billion in 2023 including $222 billion in Medicare and Medicaid costs. Without effective therapeutics and/or prophylactics, these costs could reach $1 trillion by 2050. Grand View Research (April, 2024) reports that the global Alzheimer's Disease therapeutics market size was valued at USD $4.05 billion in 2022 and is expected to grow at a compound annual growth rate (CAGR) of 19.99% from 2023 to 2030 to USD $20.88 billion in 2030. The rising prevalence of Alzheimer's Disease (AD) and approval of disease-modifying therapies are expected to fuel market growth.

The launch of novel agents that affect disease progression will transform the AD market, which is currently comprised solely of palliative (symptomatic) therapies. The ability to arrest progression of or cure AD will be the optimal outcome from the use of these novel targeted nanosomes.

Current Alzheimer's Disease Therapeutics. Currently, there are few therapeutic interventions for treating or curing AD; most of the available FDA-approved drugs are only palliative for the symptoms of AD. Prior to 2023, the FDA approved only 5 drugs to treat AD including cholinesterase inhibitors (donepezil, galantamine, rivastigmine, tacrine) which enhance deficient cholinergic signaling and Memantine, an NMDA (N-methyl-D-aspartate) antagonist, which reduces Ca²⁺ influx, oxidant stress and neural excitotoxicity. None of these agents cures the disease and all have potentially serious side effects.

In 2023, the FDA granted accelerated approval Biogen/Eisai's therapeutic drug Leqembi (lecanemab) for mild cognitive impairment or the mild dementia stage of AD. The drug is the second in the class of new medications (β-amyloid targeting antibodies) approved by the FDA targeting the fundamental pathophysiology of the disease. In 2021, the FDA approved another Biogen/Eisai drug Aduhelm (aducanumab) for its ability to bind and eliminate β-amyloid protein clusters. This drug was removed from the market by the developers because of several factors including performance, side-effects, cost and customer (clinician) acceptance. Some of the drawbacks of Leqembi are similar to those of Aduhelm—limited and mixed clinical trial data, side effects including brain swelling and bleeding, and high cost. The FDA's accelerated approval for Leqembi means that further studies may be necessary to confirm the drug's clinical benefits; the FDA denied accelerated approval of Eli Lily's adonanemab that also targets amyloid protein clusters, requesting more data.

Alpha (α)-Secretase, a Novel Approach for Treating AD. Alpha (α)-secretase is an enzyme in the neuronal pathway that positively influences amyloid precursor protein (‘APP’) processing. β-secretase and γ-secretase cooperate to cleave APP to form insoluble amyloid plaques (Aβ) that set-in motion tau fiber entanglement. In contrast, α-secretase cleaves APP into a harmless and much more soluble product, called ‘sAPP-α’, that actually supports new synapse formation. sAPP-α is safely and readily cleared from the brain. Thus, unlike current strategies which seek to suppress Aβ plaque formation by minimizing β- and γ-secretase activities, activation of the normal α-secretase pathway effectively degrades the substrate for β-amyloid generation, and at the same time leads to beneficial amyloid precursor processing to both prevent and reduce Aβ plaques and tau fibers in AD, and increase the making of synapses or ‘synaptogenesis’.

Bryostatin-1 and other Bryostatins (Bryoids): Novel α-Secretase Modulators and Potential AD Therapeutics. Bryoids are neuroprotective in AD via activation of α-secretase, directly and via novel protein kinase C (PKC) isoforms, to cleave APP and form its soluble and harmless relative, sAPP-α.

Nanoparticles can significantly improve the delivery of novel therapeutics such as Bryoids to the central nervous system (CNS). Precision manufacturing of these nanoparticles allows for the precise delivery of Bryoid reducing local therapeutic concentrations and toxicities while improving therapeutic index, significantly reducing side-effects and cost of treatment. The manufacturing of nanosomes in a microgravity environment brings about a novel manufacturing technique that is dominated by interfacial tension versus gravity forces. Microgravity production of nanoparticles is vastly superior to manufacturing in the normal 1g gravity environment of the Earth, mainly because of the size of the particles that are generated.

Nanoparticle size is related to patient uptake and biological half-life. Therapeutic products generated in microgravity environments are smaller and more uniform compared to those produced in 1g normal gravity. Smaller nanoparticles size increases penetration rate and thus reduces the cost per dose. Furthermore, microfine nanoparticles have superior transport properties in microsprays and much greater surface area for uptake. Thus, the manufacture of rare therapeutics such as Bryoids in microgravity environments conserves these scarce source materials, making these therapeutics more economical and available to larger numbers of patients. Embodiments of the present invention form nanosomes having an average diameter between 0.001 and 1,000 nanometers and, most preferably, 0.001 and 200 nanometers. The narrow diameter range of the nanosomes obtained with the disclosed methods using microgravity environments is unusual and surprising. The disclosed liposomal nanoparticles are not coated with any polymer, as in alternative technologies. No coating of any kind is required to stabilize the disclosed nanoparticles.

Bryostatin-1, a macrolide lactone, was first isolated from the bryozoan Bugula neritina by Pettit et al. (1984) and more recently characterized as a product of a bacterial symbiont of the bryozoan (Davidson et al., 2001). Several groups (Yi et al., 2012) have demonstrated that protein kinase C (PKC) activation is an important means of ameliorating AD pathophysiology and cognitive impairment.

Recently, the inventor discovered and identified a more potent analog of Bryostatin-1 (C₄₅H₆₂O₁₇; MW=874.4), called Bryoid 10, which is about 250% more neuroprotective by α-secretase activation via novel PKC isoforms, down-regulation of pro-inflammatory and angiogenic processes and the substitution of β-amyloid for its soluble and harmless relative, sAPP-α (Castor, European, Chinese and Japanese Patents, 2018 and 2019).

The inventor has also discovered and identified a second Bryoid composition having a molecular weight of approximately 910-912 Amu (Mass+Sodium) having a purity of approximately 50% to a crystal forming purity. The second Bryoid composition can also be characterized as a Bryoid compound having a molecular weight of approximately 888-890 Amu (monoisotopic mass) having a purity of approximately 50% and a crystal forming purity. The second Bryoid composition has a measured mass plus sodium of 911.5 Amu and a measured monoisotopic mass of 888.9 Amu. The detailed discussion which follows will refer to this Bryoid as B12.

The inventor has also discovered and identified a third Bryoid composition having a molecular weight of approximately 868-870 Amu (Mass+Sodium) having a purity of approximately 50% to a crystal forming purity. The third Bryoid composition can also be characterized as a Bryoid compound having a molecular weight of approximately 846-848 Amu (monoisotopic mass) having a purity of approximately 50% and a crystal forming purity. The third Bryoid composition has a measured mass plus sodium of 869.5 Amu and a measured monoisotopic mass of 846.6 Amu. The detailed discussion which follows will refer to this Bryoid as B14B.

Encapsulating Bryoids in nanosomes has the potential to reduce toxicity by reducing the uptake of the drug by unintended organs such as liver and kidneys since less free drug is available in the circulatory system. Encapsulation would also delay the clearance of the drug thus increasing its half-life. While liposomes have been used successfully to deliver and improve the therapeutic effect of several anticancer and antifungal drugs, there are no FDA-approved liposomal preparations that encapsulate drugs for Alzheimer's disease.

Our in vitro studies have shown that Bryostatin-1 and related ‘Bryoids,’ activate PKC-δ and PKC-ε isoforms, and are highly active Alzheimer's disease treatments that enhance APP processing at concentrations orders of magnitude lower than conventional APP modulators (Yi et al., 2012). We have conducted extensive in vivo pharmacokinetic and pharmacodynamics studies with radio-labeled Bryostatin-1. Our in vivo studies have shown that Bryostatin-1 is intranasally-active and orally active, and rapidly restores cognitive performance in AD-transgenic mice (Schrott et al., 2015). Our studies suggest that both Bryostatin-1 and other Bryoids such as Bryoid-10, Bryoid-12 and Bryoid-14B are excellent candidates for translation into human clinical trials and potential therapeutics for AD therapy.

The International Space Station (ISS) is an interplanetary facility in which nanoencapsulation technologies like those anticipated for microfined nanoparticles can be achieved in sufficient yields to allow testing of these relative ‘exotic’ AD therapeutics. The role of the ISS is to allow a stable manufacturing platform to create, capture and test these nanoparticles. Persistent microgravity reduces the rate at which nanoparticles aggregate and reduces the size of nanoparticles formed under microgravity. The formation and maintenance of smaller nanoparticles promotes superior pharmacology and biochemistry, justifying the nanoencapsulation of Bryoids as well as other highly precious pharmaceuticals whose appropriate packaging could unlock their commercial and clinical potential. Microgravity affects the size and character of the nanosomes, which provides major advantages in the manufacture of rare therapeutics and the efficient use of rare source materials.

Aspects of the present invention employ materials known as supercritical, critical or near-critical fluids. A material becomes a supercritical fluid at conditions which equal or exceed both its critical temperature and critical pressure. The parameters of critical temperature and critical pressure are intrinsic thermodynamic properties of all sufficiently stable pure compounds and mixtures. Carbon dioxide, for example, becomes a supercritical fluid at conditions which equal or exceed its critical temperature of 31.1° C. and its critical pressure of 72.9 atm (1,070 psia). In the supercritical fluid region, normally gaseous substances such as carbon dioxide become dense phase fluids which have been observed to exhibit greatly enhanced solvating power. At a pressure of 3,000 psig (204 atm) and a temperature of 40° C., carbon dioxide has a density of approximately 0.845 g/cc and an interfacial tension approaching zero dynes per cm, and behaves much like a nonpolar organic solvent having a dipole moment of zero Debyes.

A supercritical fluid displays a wide spectrum of solvation power as its density is strongly dependent upon temperature and pressure. Temperature changes of tens of degrees or pressure changes by tens of atmospheres can change a compound solubility in a supercritical fluid by an order of magnitude or more. This feature allows for the fine-tuning of solvation power and the fractionation of mixed solutes. The selectivity of nonpolar supercritical fluid solvents can also be enhanced by addition of compounds known as modifiers (also referred to as entrainers or cosolvents). These modifiers or cosolvents are typically somewhat polar organic solvents such as acetone, ethanol, methanol, methylene chloride or ethyl acetate. Varying the proportion of modifier allows wide latitude in the variation of solvent power.

In addition to their unique solubilization characteristics, supercritical fluids possess other physicochemical properties which add to their attractiveness as solvents. They can exhibit liquid-like density yet still retain gas-like properties of high diffusivity and low viscosity. The latter increases mass transfer rates, significantly reducing processing times. Additionally, the ultra-low surface tension of supercritical fluids allows facile penetration into microporous materials, increasing extraction efficiency and overall yields.

A material at conditions that border its supercritical state have properties that are similar to those of the substance in the supercritical state. These so-called “near-critical” fluids are also useful for the practice of this invention. For the purposes of this invention, a near-critical fluid is defined as a fluid which is (a) at a temperature between its critical temperature (Tc) and 75% of its critical temperature and at a pressure at least 75% of its critical pressure, or (b) at a pressure between its critical pressure (Pc) and 75% of its critical pressure and at a temperature at least 75% of its critical temperature. In this definition, temperature and pressure are defined on absolute scales, e.g., Kelvin and psia. To simplify the terminology, materials which are utilized under conditions which are supercritical, near-critical, or exactly at their critical point with or without small molar concentrations of polar cosolvents are jointly referred to as “SuperFluids™” or referred to as “SFS.”

SuperFluids™ can be used for the encapsulation of hydrophilic molecules such as siRNA and hydrophobic molecules such as Bryoids and combinations of hydrophilic and hydrophobic molecules in phospholipid nanosomes (small, uniform liposomes). Bryoids are quite hydrophobic and are packaged in the lipid bilayer. The nanosomal formulation of the Bryoid drugs results in reduced systemic toxicity, due to the masking of the cytotoxic effects of Bryoids. By increasing residence time in the circulatory system, the nanosomes increase therapeutic efficacy of Bryoids. Optionally, pegylated phospholipids provide steric hindrance that further increase residence time and therapeutic efficacy as is done with Doxil®, liposome encapsulated doxorubicin. Furthermore, phospholipids linked with specific antibodies or ligands are used to target the encapsulated Bryoids to specific areas of the brain. Such smart targeting further reduces toxicities associated with Bryoids while increasing efficacy and therapeutic index.

SuperFluids™, under similar conditions to those used for making nanosomes, have been shown to exhibit significant microbicidal and virucidal effects that contribute to the sterility of the final formulation during manufacturing.

Conventional processes for the encapsulation of hydrophobic drugs utilize many processing steps and require large quantities of organic solvents. These processes are very time consuming, costly and inefficient. Generally, such phospholipid liposomes have a wide dispersion of particle size. In addition, the exposure of therapeutic agent to the organic solvent may adversely affect the integrity of the final product. Other conventional processes for the encapsulation of hydrophilic drugs into phospholipid liposomes utilize high pressure homogenization that requires a significant amount of recycling, generates heat with every pass through the homogenizer, and could be contaminated with heavy metal particles. These conventional processing methods do not provide sterility and may also compromise sterility.

Embodiments of the present invention address these problems inherent in the prior art with the application of supercritical, critical or near-critical fluids with or without a cosolvent or modifier. Embodiments of the present invention are enhanced in the microgravity environment of Space.

Embodiments of the present invention are directed to methods of using supercritical fluids for encapsulating hydrophobic drugs in phospholipid liposomes in a microgravity environment. The size, uniformity and integrity of such liposomes make such liposomes ideal for containing therapeutic drugs such as Bryoids and other products for ‘orphan’ and chronic diseases such as cancer, HIV and Alzheimer's disease. These methods require reduced processing steps, time and preparation costs.

One embodiment of the present invention is a method of making phospholipid nanosomes comprising the steps of providing a phospholipid solution of dissolved in a first fluid. The first fluid consists of a supercritical, critical or near-critical fluid with or without a cosolvent or modifier. The phospholipid-enriched phospholipid solution is then mixed with a solution of hydrophobic drug in first fluid or an alcoholic solution. Next, the phospholipid and hydrophobic drug solution is depressurized as said phospholipid and hydrophobic drug solution exit one or more orifices in the presence of a low solubility fluid. The low solubility fluid has low volatility and the phospholipid and hydrophobic drug materials are in concentrations which exceed the solubility of the phospholipid and hydrophobic materials in the low solubility fluid. The phospholipid and hydrophobic drug materials form liposomes containing the hydrophobic drug, and the first fluid is removed during depressurization.

Embodiments of the present invention feature the formation of nanosomes having an average diameter between 0.001 and 1,000 nanometers and, most preferably, 0.001 and 200 nanometers. The narrow range of diameter of the nanosomes that can be attained with the present method is unusual and surprising.

Preferably, the phospholipid and hydrophobic drug solution is depressurized to ambient pressure in a microgravity environment.

A preferred phospholipid is selected from one or more of the group of synthetic and derivatized phospholipids, including phosphatidylcholine (PC), phosphatidylglycerol (PG), phosphatidylserine (PS), dimyristoylphosphatidylcholine (DMPC), dimyristoylphosphatidyl-glycerol (DMPG), phosphatidylethanolamine (PE) and polyethylene conjugated distearylphosphatidylethanolamine (either DSPE-PEG₂₀₀₀ or DSPE-PEG₃₅₀₀), cationic lipids such as MVL5 (N1-[2-((1S)-1-[(3-aminopropyl)amino]-4-[di(3-amino-propyl)amino]butylcarboxamido)ethyl]-3,4-di [oleyloxy]-benzamide) and α-tocopherol (vitamin E), a common non-toxic dietary lipid, as an anti-oxidant. The phospholipid may contain specific antibodies or ligands for specific diseases of the mind and body such as cancer, HIV and Alzheimer's disease.

Preferred first fluids comprise propane, fluorohydrocarbons, nitrous oxide, ethylene, ethane and carbon dioxide. The first fluid may also contain cosolvents or modifiers. Preferred modifiers are ethanol, methanol, propanol, butanol, methylene chloride, ethyl acetate and acetone. A preferred temperature and pressure for a SuperFluids™ comprising propane are a temperature in the range of 10 to 60° C. and a pressure in the range of 1,000 to 5,000 psig.

The low solubility fluid, preferably, comprises an aqueous solvent, such as distilled water or a buffer such as PBS. Preferably, the low solubility fluid has a chemical agent such as sucrose and trebalose for stabilizing the liposomes.

### BRIEF DESCRIPTION OF THE DRAWINGS

FIG. 1 depicts in schematic form an apparatus embodying features of the present invention.

FIG. 2 shows HEK-293e cells stimulated for 24 h with Bryostatin-1 at different concentrations. 10⁻⁸M Bryostatin-1 significantly increased the release of sAPP-α (measured as an increase in absorbance at 620 nm, n=3, avg+SD).

FIG. 3 shows alpha-secretase activation as a function of time for two concentrations of Bryosomes™ interacting with HEK-293e cells.

FIG. 4 shows a schematic of cryotube bioreactor.

FIG. 5 shows standard curves for the alkaline phosphatase activity assays.

FIG. 6 shows time course of sAPP-α activity in HEK-293e cells.

### DETAILED DESCRIPTION OF PREFERRED EMBODIMENTS

As stated earlier, microgravity, microgravity environment, or microgravity conditions are defined as a very low gravity force of less than 1×10⁻³×g. The units for microgravity are expressed as a multiplier of normal terrestrial gravity (g) as measured on the surface of the earth. This definition of microgravity as less than 1×10⁻³×g is supported by the National Aeronautics and Space Administration of the United States. Microgravity may be achieved in certain trajectories of rockets. Microgravity may be achieved in reduced-gravity aircraft flying in a parabolic trajectory. A reduced gravity aircraft is a type of fixed-wing aircraft that provides brief near-weightless environments for training astronauts, conducting research, and making gravity-free movie shots. Even longer periods of microgravity may be obtained in orbital or suborbital spacecraft such as the NASA space shuttle. Relatively indefinite periods of microgravity may be obtained in permanent or semi-permanent orbital space stations such as the International Space Station (ISS) and in orbital satellites, such as geosynchronous orbital satellites. Microgravity may be obtained in interplanetary space probes and spacecraft.

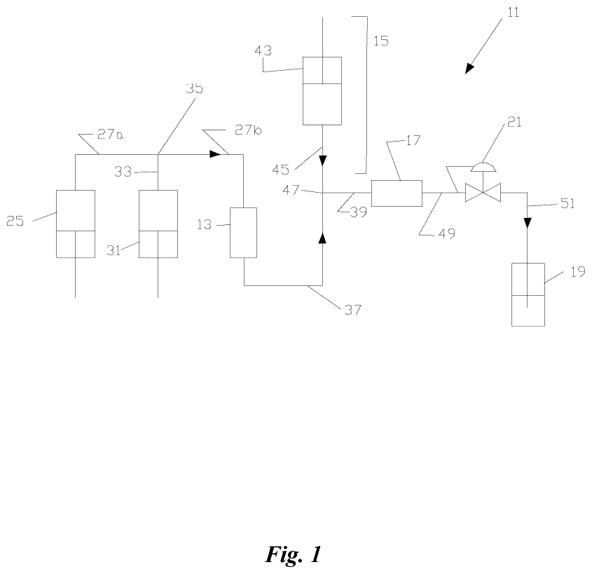

The present method and apparatus will be described with respect to FIG. 1 which depicts in schematic form a phospholipid liposome apparatus, generally designated by the numeral 11. The phospholipid nanosomes apparatus is comprised of the following major elements: a phospholipid vessel 13, a hydrophobic drug injection assembly 15, an admixture chamber 17, a depressurization vessel 19, and a back-pressure regulator 21.

Phospholipid vessel 13 is in fluid communication with a SuperFluids™ syringe pump 25 via conduits 27a and 27b. SuperFluids™ pump 25 is in fluid communication with a source of SuperFluids™ (not shown).

Phospholipid vessel 13 is also in fluid communication with a coslovent syringe pump 31 via conduit 33 which intersects with conduit 27a and 27b at junction 35. Cosolvent syringe pump 31 is in communication with a source of modifiers and/or entrainers (not shown).

Phospholipid vessel 13 is loaded with phospholipid. And, phospholipid vessel receives SuperFluids™ from SuperFluids™ pump 25 via conduits 27a and 27b. Phospholipid vessel 13 receives coslovents and/or entrainers from Cosolvent pump 31 via conduit 33. Phospholipid is dissolved in the SuperFluids™ and modifier to form a phospholipid solution.

Phospholipid vessel 13 is in fluid communication with admixture chamber 17 via conduits 37 and 39. Admixture chamber 17 is also in fluid communication with hydrophobic drug injection assembly 15. Hydrophobic drug injection assembly 15 comprises hydrophobic drug syringe pump 43, a source of hydrophobic drug material (not shown) and conduit 45. Hydrophobic drug syringe pump 43 is in communication with a source of hydrophobic drug material and pressurizes and compels such material through conduit 45. Conduit 45 is in communication with admixture chamber 17 via conduit 39 which intersects conduit 45 at junction 47. Preferably junction 47 is a mixing “T”.

Admixture vessel 17 is in the nature of an inline mixer and thoroughly mixes incoming streams from the phospholipid vessel 13 and hydrophobic drug injection assembly 15. Admixture vessel 17 is in communication with back-pressure regulator 21 via conduit 49. Back-pressure regulator 21 is connected via conduit 51 to a nozzle defining one or more orifices which discharge into depressurization vessel 19. Preferably back-pressure regulator 21 controls pressure and decompression rates, and the size of the nozzles controls bubble and particle sizes.

The operating pressure of the system can be preset at a precise level via a computerized controller (not shown) that is part of the syringe pumps. Temperature control in the system is achieved by enclosing the apparatus 11 in ¼″ Lexan sheet while utilizing a Neslab heating/cooling system coupled with a heat exchanger (not shown) to maintain uniform temperature throughout the system.

In a typical experimental run, phospholipid raw materials were first packed into phospholipid vessel 13. SuperFluids™ and cosolvents such as ethanol were charged into the SuperFluids™ syringe pumps 25 and 31, respectively, and brought to the desired operating pressure. An ethanolic solution of hydrophobic drug is charged into bioactive syringe pump 43.

The system was then pressurized with the SuperFluids™ (supercritical fluid (SCF) and cosolvent) via SuperFluids™ syringe pump 25 to the pressure level equal to that set-in modifier syringe pump 31 and hydrophobic drug syringe pump 43, and maintained at this level with the back-pressure regulator 21. The dynamic operating mode for all pumps was set so that each pump can be operated at its own desired flow rate. The SuperFluids™ stream flowed through the phospholipid vessel 13, dissolved phospholipid and contacted the hydrophobic drug stream at junction 47. The mixture of SuperFluids®, hydrophobic drug and phospholipid materials was then passed through admixture chamber 17 for further mixing. Finally, the mixed solution entered back-pressure regulator orifice nozzle 21 and conduit 51, and was injected into a 10% sucrose solution containing hydrophilic drug in the depressurization vessel 19. As a result of supercritical fluid decompression, phospholipid nanosomes containing hydrophobic drug and/or hydrophilic drug are formed in the 10% sucrose solution and the expanded supercritical fluid exited the system via a vent line on the depressurization vessel 19.

Depressurization is performed in a normal gravity environment. The phospholipid nanosomes are then frozen and lyophilized. The lyophilized powder is reconstituted, frozen and transported to a microgravity environment in Space wherein the frozen solution is thawed and mixed forming phospholipid nanosomes. The nanosomes are then re-frozen and reconstituted in a normal terrestrial gravity environment of approximately 1g.

Depressurization is performed in a microgravity environment. The phospholipid nanosomes are then frozen and lyophilized. The lyophilized powder is reconstituted in a microgravity or normal gravity environment forming phospholipid nanosomes.

### EXAMPLES

### Example 1: Encapsulation of Bryostatin-1 in Phospholipid Nanosomes (SPX-I-01)

In SPX-I-01, Bryosomes (nanosomes containing Bryostatin-1) were made using PC 16:0/16:0 DPPC and PEG-2000 DSPE for targeted molar ratio of 20:1 using SFS Propane:Ethanol::80:20 at P=3,000 psig and T=40° C., 0.01″ injector into 10% sucrose buffer at 4° C. and pH=7.0, a feed of ethanol containing Bryostatin-1 (0.00867 mg/mL) and MPEG-2000-DSPE (5.245 mg/mL), and co-injection ratio (SFS:Feed) ratio of 1.0. The results of SPX-I-01 are listed in Table 1.

TABLE 1
Chemical Content and Physical Size
of SPX-I-01 containing Bryostatin-1

| | | Unimodal | SDP |
|---|---|---|---|
| | Concentration | Particle Size | Particle Size |
| Sample | (μg/mL) | (nm) | (nm) |
| SPX-I-01-03 | 1.662 | 221 | NA |
| SPX-I-01-03-00-01-01 | 1.567 | 89.8 | RW |

NA - Not Available;
RW - Range Warning

Unimodal particle size provides a measure of the sample particle mean size and a measure of the polydispersity or breadth of the particle size distribution. The unimodal analysis extracts the first two moments of size distribution; that is, the mean particle size and the standard deviation. The results are only valid if the true sample distribution is a log Gaussian or close approximation. The SDP (Size Distribution Processor) particle size analysis obtains the actual distribution of particle sizes rather than only the first two moments and is independent of the log Gaussian assumption. Range warning (RW) occurs if the SDP particle size is less than 3 nm or greater that 3 microns.

SPX-I-01-03 was the first and major fraction (01) produced that was degassed by Recon method (03), vortexing for 3×30 seconds, light sonication for 10 minutes and degassed under vacuum for 6 minutes.

SPX-I-01-03-00-01-01 was SPX-I-01-03 which was frozen at −80° C. and lyophilized over 48 hours (00), then reconstituted by vortexing for 3×30 seconds and degassed under vacuum for 6 minutes (01), and then sterile filtered using a Nalgene 50 mL 0.22 μm filtration unit (01).

The sterile SPX-1-01-03-00-01-01 product contained 1.567 g/mL and had a unimodal; particle size of 89.8 nm.

### Example 2: Encapsulation of Bryostatin-1 in Phospholipid Nanosomes (SPX-I-02)

In SPX-I-02, Bryosomes (nanosomes containing Bryostatin-1) were made using PC 16:0/16:0 DPPC and PEG-2000 DSPE for targeted molar ratio of 20:1 using SFS Propane:Ethanol::80:20 at P=3,000 psig and T=40° C., 0.01″ injector into 10% sucrose buffer at 4° C. and pH=7.0, a feed of ethanol containing Bryostatin-1 (0.000667 mg/mL) and MPEG-2000-DSPE (4.051 mg/mL), and co-injection ratio (SFS:Feed) ratio of 1.0. The difference between SPX-I-01 and SPX-I-02 is in the Bryostatin-1 concentration that is about 13 times lower in SPX-I-02. The reduction or scale-down was performed to evaluate the nanoencapsulation of low concentrations of Bryostatin-1 required for the in vitro analyses described in Example 3. The results of SPX-I-02 are listed in Table 2.

TABLE 2
Chemical Content and Physical Size
of SPX-I-02 containing Bryostatin-1

| | | Unimodal | SDP |
|---|---|---|---|
| | Concentration | Particle Size | Particle Size |
| Sample | (μg/mL) | (nm) | (nm) |
| SPX-I-02-01-03 | 0.232 | 221 | 295 |
| SPX-I-02-01-03-00-03-01 | 0.0873 | 138 | 120 |

SPX-I-02-01-03 was the first and major Fraction (01) produced that was degassed by Recon method 03, vortexing for 3×30 seconds, light sonication for 10 minutes and degassed under vacuum for 6 minutes.

SPX-I-02-01-03-00-03-01 was SPX-I-02-01-03 which was frozen at −80° C. and lyophilized over 48 hours (00), then reconstituted by vortexing for 3×30 seconds and degassed under vacuum for 6 minutes (03), and then sterile filtered (01) using a Nalgene 50 mL 0.22 μm filtration unit.

The sterile SPX-I-02-01-03-00-03-01 product contained 0.0873 μg/mL (˜ 10⁻⁷M) and had a particle size of 138 nm.

### Example 3: HEK-293E Cell Model for Amyloid Precursor Protein (APP) Processing

The HEK-293e cell line was optimized for studying α-secretase in vitro. HEK-293e is a human embryonic kidney cell line that has been transfected to express an alkaline phosphatase tagged amyloid precursor protein, which allows rapid evaluation of sAPP-α in the supernatant from cells in culture in response to a stimulus, e.g., Bryostatin-1.

HEK-293e cells are grown to confluency in T75 dishes in DMEM (Dulbecco's Modified Eagle Medium) and 10% FCS (Fetal Calf Serum) with 2 mM L-glutamine and pen-strepamphotericin. Supplements are needed to maintain plasmid expression: (1) Hygromycin stock solution=100× solution=4 mg/mL, final concentration=40 μg/mL (50 mg/12.5 mL makes 4 mg/mL); (2) Puromycin stock solution=100× solution=30 μg/mL stock, final concentration=0.3 μg/mL. Wait for 24-48 hours. All studies used phenol Red Free DMEM medium with 2% FCS. This must be heated to 65° C. for 30 minutes to inactivate endogenous alkaline phosphatase and cooled prior to making up test solutions.

For test solutions, remove 10% FCS growth medium and replace with 1 mL phenol red free DMEM medium+2% FCS per tube (various drug concentrations and control), and freeze cells in cryotubes. Thaw cryotubes and then: (1) Incubate cells at 37° C. for different time points; (2) Centrifuge tube at 1×g for 5 minutes and refreeze tubes to isolate culture supernatants; (3) Thaw tubes and transfer supernatants to new tube: (4) Make up substrate—Phos Blue (KPL Cat #50-88-00) according to manufacturer's instructions; (5) Transfer 100 L of each sample and add 100 μL of substrate to 96 well plates; (6) Read immediately—record absorbance at 620 nm; and (7) Reacts quickly so record absorbance every 15 minutes for 60 minutes.

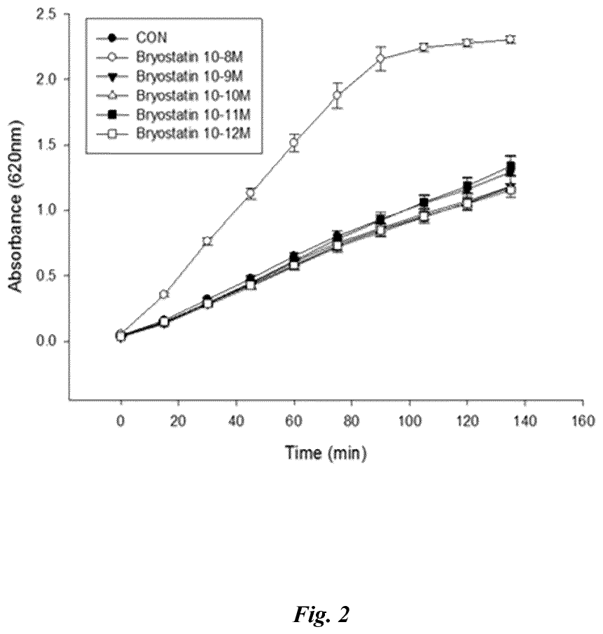

FIG. 2 shows that 10⁻⁸M Bryostatin-I strongly induces sAPP-α release, but that lower concentrations do not affect release. This indicates that processing in this cell line is less sensitive to Bryostatin-1 than in SH-SY5Y neuroblastoma cells, but is able to provide important and rapid information on Bryostatin-1 at higher concentrations.

### Example 4: Encapsulation of Bryostatin-1 in Phospholipid Nanosomes (SPX-I-08)

In SPX-I-08, Bryosomes™ (nanosomes containing Bryostatin-1) were made by SuperFluids™ CFN™ technology using PC 16:0/16:0 DPPC and PEG-2000 DSPE for targeted molar ratio of 20:1 using SFS Propane:Ethanol::80:20 at P=3,000 psig and T=40° C., 0.01″ injector into 10% sucrose buffer at 4ºC and pH=7.0, a feed of ethanol containing Bryostatin-1 (0.00667 mg/mL) and MPEG-2000-DSPE (4.051 mg/mL), and co-injection ratio (SFS:Feed) ratio of 1.0. This experiment was conducted to produce materials for flight operations. The results of SPX-1-08 are listed in Table 3.

TABLE 3
Chemical Content and Physical Size
of SPX-I-08 containing Bryostatin-1

| | | Unimodal | SDP |
|---|---|---|---|
| | Concentration | Particle Size | Particle Size |
| Sample | (μg/mL) | (nm) | (nm) |
| SPX-I-08-01-03-00-03 | 0.309 | 215 | 134 |
| SPX-I-08-01-03-00-03-01 | 0.323 | 196 | 205 |

SPX-I-08-01-03-00-03 was the first and major fraction (01) produced that was degassed by Recon method (03), vortexing for 3×30 seconds, light sonication for 10 minutes and degassed under vacuum for 6 minutes, frozen at −80° C. and lyophilized over 48 hours (00) and the reconstituted with DI water by Recon method 03. This product was then sterile filtered (01) using a Nalgene 50 mL 0.22 μm filtration unit to produce SPX-I-08-01-03-00-03-01.

### Example 5: Encapsulation of Bryostatin-1 in Phospholipid Nanosomes (SPX-I-09)

The objective of SPX-I-09 was to dilute SPX-I-08-01-03-00-03-01 into four different molar concentrations to simulate the samples that were aliquoted and packaged for shipment to the ISS and for pre-flight testing. These Bryostatin-1 concentrations were 4×10⁻⁸ M. 10⁻⁸ M, 2×10⁻⁷ M and 0.5×10⁻⁷ M. The 4×10⁻⁸ M and 2×10⁻⁷ M aliquots are for the cryotubes with cells and media (which experience a 1:4 dilution on mixing), and the 10⁻⁸ M and 0.5×10⁻⁷ M aliquots are for cryotubes without cells and media (Table 4). An additional objective was to select the best molar concentration of Bryostatin-1 for testing on the ISS.

TABLE 4
Chemical Content and Physical Size of SPX-I-09 Bryosomes ™

| | Unimodal | SDP |
|---|---|---|
| Bryostatin-1 Concentration | Particle Size | Particle Size |
| (nM) | (nm) | (nm) |
| 10 | 165 | RW |
| 40 | 155 | RW |
| 50 | 245 | 139 |
| 200 | 185 | 197 |

RW - Range Warning

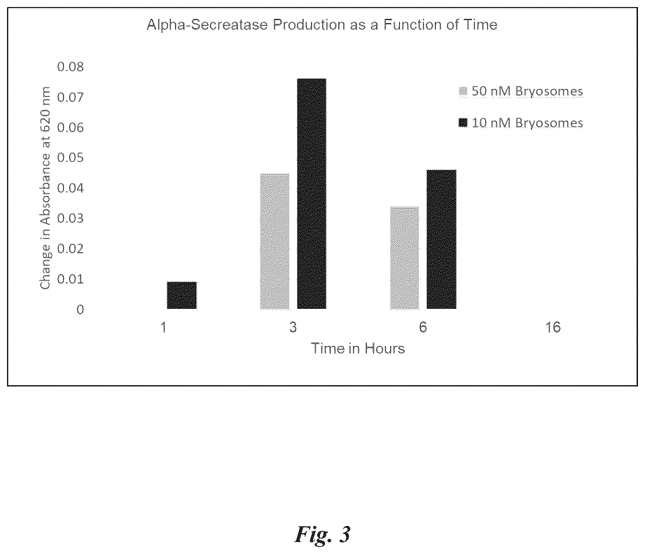

After freezing Bryosomes™, HEK-293e cells and cell culture media at −80ºC and thawing after 1, 3, 6 and 16 hours, the samples were analyzed for α-secretase production (FIG. 3). The results indicate that best results were obtained at 10 nM (10⁻⁸ M). This result is consistent with the results of SPX-1-07 and prior results with Bryostatin-1. Thus, the concentration of 10⁻⁸ M Bryosomes™ after mixing was targeted for the Space mission.

### Example 6: Encapsulation of Bryostatin-1 in Phospholipid Nanosomes (SPX-1-11 and SPX-1-12)

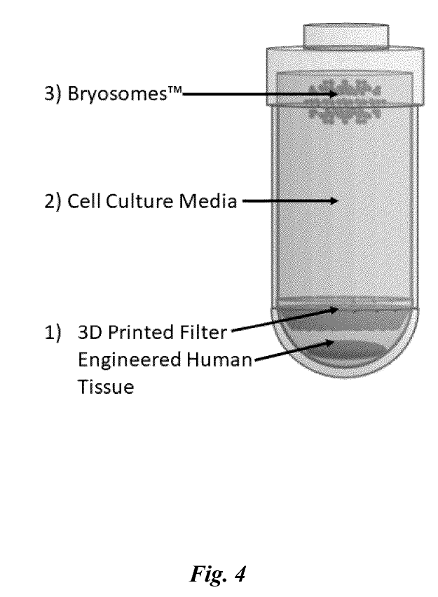

For SPX-I-11 and SPX-I-12, cryotubes containing 1 mL of approximately 100,000 HEK-293e cells were prepared. The HEK-293e cells were then frozen at −80° C. 2 mL of cell culture media was then added on top of the cells and frozen at −80° C. 1 mL of 4×10⁻⁸ M Bryosomes™ was then added to the cryotube and frozen at −80° C. forming a 3-layer ice cream cake shown as FIG. 4. Cryotubes were also prepared containing 4 mL of 10⁻⁸ M Bryosomes™. Double Ziploc packed experimental packets were prepared and labeled Bag 1, Bag 2, Bag 4 and Bag 8. Within each packet, there are six tubes: 3 tubes containing HEK-293e cells with Bryosomes™ at a final concentration of 10⁻⁸ M (after mixing) and 3 tubes containing Bryosomes™ at 10⁻⁸ M.

Four double Ziploc packed bags were shipped to STaARS, Houston, TX on dry ice, then transported to NASA on dry ice and transferred to the International Space Station (ISS) on NG-11, previously known as OA-11, is the twelfth flight of the Northrop Grumman robotic resupply spacecraft Cygnus and its eleventh flight to the International Space Station under the Commercial Resupply Services contract with NASA. Bioscience-11 on Cygnus NG-11 was launched to the ISS on Apr. 17, 2019 at 16:46:07 pm EST. NG-11 docked onto the ISS on the morning of Friday, Apr. 19, 2019. During this chain of custody, the samples were either maintained on dry ice or at −80° C.

On the ISS, these cells were thawed or ‘revived’ from cryopreservation in the specially prepared reaction tubes in the 4 bags, shaken gently, and allowed to react for time points up to 8 hours at 37ºC Then Bag 1. Bag 2, Bag 3 and Bag 4 was respectively refrozen at 1, 2, 4 and 8 hours post-thawing. Bags of tubes were held frozen until re-entry and transported to Earth for analysis to evaluate time dependent activation of alpha-secretase in response to Bryosomes™ at 10⁻⁸ M in a microgravity environment of Space.

The four (4) Bags of cryotube samples on dry ice were received by Aphios Corporation on Nov. 19, 2019 frozen and in good shape. The samples were shipped from STaARS, Houston, TX on Nov. 18, 2019. STaARS reported the samples were stored at −80° C. during the period of their return to earth (unberthing date Aug. 6, 2019) and shipment to Aphios. On receipt at Aphios, the samples were stored in a −80° C. freezer in BSL-2 Laboratory 101. On Nov. 26, 2019, the sample bags were taken out of the −80° C. freezer and opened to remove the frozen samples. The results of the particle size and HPLC analyses are listed in Table 5. The SPX-I-11 sample is the analysis of the product used on Earth in preparation of the cryotubes containing 10 nM Bryosomes™. This sample was not transported to the ISS.

TABLE 5
Particle Size and HPLC Analyses of SPX-I-12 Samples Returned from ISS

| | | | Unimodal | SDP | |
|---|---|---|---|---|---|
| | Time | | Particle Size | Particle Size | Bryostatin-1 |
| Samples | (h) | Composition | (nm) | (nm) | (nM) |
| | | 100 nm Standard | 114 | 97.2 | NA |
| SPX-I-11 | 0 | 10 nM Bryosomes ™ | 259 | 89 | 350.114 |
| | | 100 nm Standard | 111 | 98.4 | NA |
| | | 100 nm Standard | 118 | 101 | NA |
| SPX-I-12 | | | | | |
| 1A | 1 | 10 nM Bryosomes ™ | 200 | 3.0; RW | 6.566 |
| 1B | 1 | 10 nM Bryosomes ™ | 142 | 12 | 6.584 |
| 1C | 1 | 10 nM Bryosomes ™ | 169 | 5.4 | 3.210 |
| 1D | 1 | HEK-293e Cells - 10 nM Bryosomes ™ | 85.9 | 3.2; RW | 4.480 |
| 2A | 2 | 10 nM Bryosomes ™ | 226 | 3.0; RW | 9.558 |
| 2B | 2 | 10 nM Bryosomes ™ | 94.8 | 3.9 | 19.625 |
| 2C | 2 | 10 nM Bryosomes ™ | 130 | 17.4 | 1.874 |
| 3D | 2 | HEK-293e Cells - 10 nM Bryosomes ™ | 139 | 6.0 | 3.482 |
| 4A | 4 | 10 nM Bryosomes ™ | 162 | 5.3 | 7.191 |
| 4B | 4 | 10 nM Bryosomes ™ | 140 | 3.0; RW | 8.168 |
| 4C | 4 | 10 nM Bryosomes ™ | 130 | 3.1; RW | 4.698 |
| 6D | 4 | HEK-293e Cells - 10 nM Bryosomes ™ | 129 | 3.7; RW | 0.689 |
| 8A | 8 | 10 nM Bryosomes ™ | 218 | 3.0; RW | 2.866 |
| 8B | 8 | 10 nM Bryosomes ™ | 183 | 4.7; RW | 4.081 |
| 8C | 8 | 10 nM Bryosomes ™ | 160 | 3.3; RW | 6.584 |
| 16D | 8 | HEK-293e Cells - 10 nM Bryosomes ™ | 79.4 | 3.0; RW | 5.592 |

NA - Not Applicable;
RW - Range Warning

In the HPLC analysis of the returned SPX-I samples in Table 5, “peaks” identified as Bryostatin-1 are either masked by the DMSO peak which also absorbs at 265 nm, the same as Bryostatin-1, or too small for accurate detection and quantification. The Bryostatin-1 concentration for each sample is the integrated signal within the retention time window for Bryostatin-1. Similarly, sample intensity for SPX-I-12 was lower than required for accurate particle size analysis. The particle sizes are presented by both unimodal particle size and SDP weight analysis.

Unimodal particle size provides a measure of the samples' particle mean size and a measure of the polydispersity or breadth of the particle size distribution. The unimodal analysis extracts the first two moments of size distribution; that is, the mean particle size and the standard deviation. The results are only valid if the true sample distribution is a log Gaussian or close approximation. The SDP (Size Distribution Processor) particle size analysis obtains the actual distribution of particle sizes rather than only the first two moments and is independent of the log Gaussian assumption. Range warning occurs if the SDP particle size is less than 3 nm or greater that 3 microns. The data indicates that the average SDP size of the nanoparticles was reduced from 89 nm to ≤3 nm.

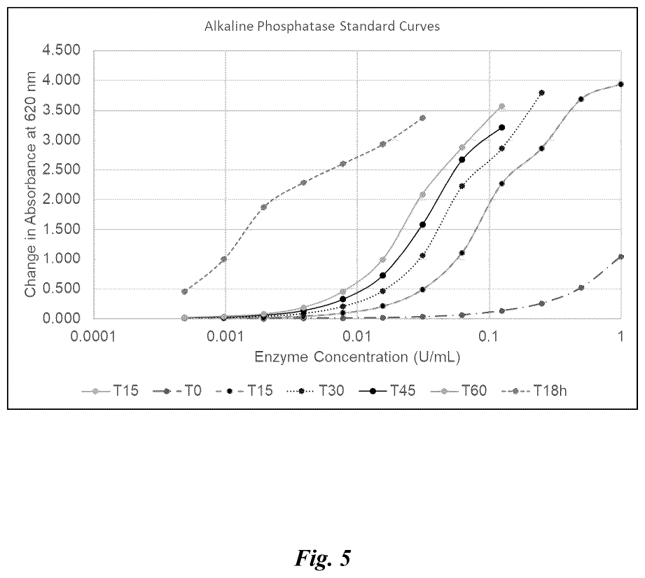

Standard Curve: The mean change in absorbance at 620 nm values for each enzyme concentration, calculated by subtracting the absorbance values for media controls from the absorbance values of each of the dilutions and then calculating the mean of the three replicates, are listed in Table 6. The mean net OD values plotted as a function of the enzyme concentration are shown in FIG. 5.

TABLE 6
Mean Change in Absorbance at 620 nm vs Alkaline Phosphatase Concentrations after Different Periods of Incubation with the Substrate

| Time of | Enzyme concentration (ug/mL) | | | | | | | | | | | | |
|---|---|---|---|---|---|---|---|---|---|---|---|---|---|
| reading | | | | | | | | | | | | | Media |
| (Mins) | 1 | 0.5 | 0.25 | 0.125 | 0.0625 | 0.03125 | 0.015625 | 0.007813 | 0.003906 | 0.001953 | 0.000977 | 0.000488 | Control |
| T0 | 0.731 | 1.038 | 0.516 | 0.255 | 0.133 | 0.063 | 0.031 | 0.015 | 0.008 | 0.005 | 0.003 | 0.005 | 0.000 |
| T15 | 3.933 | 3.683 | 2.854 | 2.264 | 1.104 | 0.488 | 0.208 | 0.094 | 0.041 | 0.019 | 0.009 | 0.007 | 0.000 |
| T30 | Too High | Too High | 3.787 | 2.853 | 2.219 | 1.055 | 0.461 | 0.203 | 0.083 | 0.036 | 0.018 | 0.011 | 0.000 |
| T45 | | | | 3.209 | 2.668 | 1.579 | 0.720 | 0.322 | 0.130 | 0.055 | 0.027 | 0.015 | 0.000 |
| T60 | | | | 3.570 | 2.869 | 2.083 | 0.988 | 0.453 | 0.185 | 0.076 | 0.038 | 0.018 | 0.000 |
| T18 h | | | | | | 3.366 | 2.924 | 2.604 | 2.282 | 1.869 | 0.994 | 0.448 | 0.000 |

The results in Table 6 and FIG. 5 indicate that for pure enzyme, the absorbance values increase with both the duration of incubation of the alkaline phosphatase enzyme with the substrate as well as the concentration of the enzyme. The curves in FIG. 5 represent the different durations of incubation with the substrate and are sigmoid shaped which is typical for enzyme reactions with saturating amounts of substrate. The linear portions of the curve are essentially parallel indicating that the activity of the enzyme is concentration dependent for each duration of incubation (i.e., time of reading).

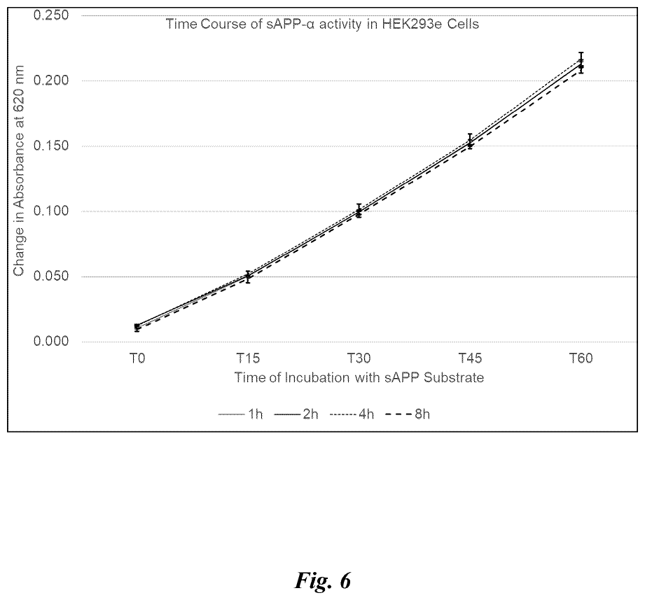

Kinetics of Alpha Secretase (sAPPP) Activity in the Supernatants of Cells Treated with Bryosomes™ in Outer Space: The mean change in absorbance at 620 nm and the standard deviations for each duration of treatment of the cells with Bryosomes™ in Space (onboard the ISS), calculated by subtracting the absorbance values for (−) cells controls from the absorbance values of each duration of treatment of the cells (+ cells) and then calculating the mean of the three replicates, are listed in Table 7. The mean change in absorbance values plotted as a function of the duration of treatment of the cell supernatants with the alkaline phosphatase substrate are shown in FIG. 6. Error bars indicating the Standard Deviations within the 3 replicates are also shown.

TABLE 7
Mean Change in Absorbance at 620 nm of Cell Supernatants
Treated with Bryosomes ™ for Different
Durations and Periods of Incubation with Substrate
Time of

| reading | Mean | | | | SD | | | |
|---|---|---|---|---|---|---|---|---|
| (Mins) | 1 h | 2 h | 4 h | 8 h | 1 h | 2 h | 4 h | 8 h |
| T0 | 0.011 | 0.013 | 0.012 | 0.009 | 0.001 | 0.001 | 0.001 | 0.002 |
| T15 | 0.050 | 0.050 | 0.052 | 0.048 | 0.001 | 0.001 | 0.002 | 0.003 |
| T30 | 0.099 | 0.099 | 0.101 | 0.098 | 0.002 | 0.002 | 0.004 | 0.002 |
| T45 | 0.153 | 0.152 | 0.155 | 0.150 | 0.002 | 0.002 | 0.005 | 0.002 |
| T60 | 0.213 | 0.213 | 0.217 | 0.208 | 0.002 | 0.003 | 0.005 | 0.003 |
| T18 h | 2.596 | 2.577 | 2.585 | 2.569 | 0.010 | 0.022 | 0.030 | 0.009 |

The results in Table 7 and FIG. 6 indicate that the change in absorbance at 620 nm increases with the duration of incubation of the substrate with the cell culture supernatants (i.e. the time of reading). However, the net absorbance values show very little or no increase with increased durations of treatment of the cells with Bryosomes™. In FIG. 6, each curve represents a duration of treatment of the cells with Bryosomes™. The four curves virtually overlap one another which indicates a lack of kinetic response of cells to Bryosomes™.

The results (FIG. 6) indicate the sAPP-α enzymatic activity response is independent of incubation time over the 60-minute test period. These results from Space were unexpected based on experiments conducted on Earth (FIG. 3).

It is intended that the matter contained in the preceding description be interpreted in an illustrative manner rather than a limiting sense.

What is claimed is:

1. A method of making phospholipid nanosomes having an average diameter between 0.001 and 200 nanometers, comprising the steps of:
a) providing a solution of a phospholipid material dissolved in a first fluid, said first fluid consisting of a supercritical, critical or near-critical fluid with or without polar cosolvent;
b) forming a solution of a hydrophobic drug in a second fluid, said second fluid is an alcohol;
c) mixing phospholipid-enriched solution in first fluid with a solution of a hydrophobic drug in in an alcohol-based solution;
d) depressurizing said phospholipid material and hydrophobic drug solution, and as said phospholipid material and hydrophobic drug exits one or more orifices in the presence of a low solubility fluid, said low solubility fluid having low volatility and said phospholipid material and hydrophobic drug in concentrations which exceed said solubility of said phospholipid material and hydrophobic drug in said low solubility fluid, said phospholipid material and hydrophobic drug forming phospholipid nanosomes having an average diameter between 0.001 and 200 nanometers and said first fluid removed during depressurization;
e) freezing and thawing said phospholipid material and hydrophobic drug solution in a microgravity environment and forming phospholipid nanosomes having an average diameter between 0.001 and 200 nanometers; and
f) wherein at least one step is performed in said microgravity environment.

2. The method of claim 1 wherein said wherein second solution for dissolving hydrophobic drug consists of a supercritical, critical or near-critical fluid with or without polar cosolvent.

3. The method of claim 1 wherein said low solubility fluid contains a hydrophilic drug.

4. The method of claim 1 wherein said phospholipid nanosomes have an average diameter between 0.001 to 20.000 nanometer.

5. The method of claim 1 wherein said phospholipid material and hydrophobic drug solution is depressurized to ambient pressure.

6. The method of claim 1 wherein said low solubility fluid is selected from the group of solvents consisting of de-ionized water, PBS, 10% sucrose and 10% trehalose solution.

7. The method of claim 1 wherein said phospholipid material is selected from one or more of the group of synthetic and derivatized phospholipids, including phosphatidylcholine (PC), phosphatidylglycerol (PG), phosphatidylserine (PS), dimyristoylphosphatidylcholine (DMPC), dimyristoylphosphatidylglycerol (DMPG), phosphatidylethanolamine (PE) and polyethylene conjugated distearylphosphatidylethanolamine (either DSPE-PEG₂₀₀₀ or DSPE-PEG₃₅₀₀), cationic lipids including MVL5 (N1-[2-((1S)-1-[(3-aminopropyl)amino]-4-[di(3-amino-propyl)amino]butylcarboxamido)ethyl]-3,4-di[oleyloxy]-benzamide) and α-tocopherol (vitamin E), a common non-toxic dietary lipid, as an anti-oxidant and derivatives thereof.

8. The method of claim 7 wherein said phospholipid may contain specific antibodies or ligands for specific diseases of the brain and body including cancer, HIV and Alzheimer's disease.

9. The method of claim 1 wherein said first fluids comprise supercritical, critical or near-critical fluid from a group consisting of propane, fluorohydrocarbons, nitrous oxide, ethylene, ethane and carbon dioxide.

10. The method of claim 1 wherein the first fluid may also contain cosolvents or modifiers from a group consisting of ethanol, methanol, propanol, butanol, methylene chloride, ethyl acetate and acetone.

11. The method of claim 9 wherein the preferred temperature and pressure for a first fluid comprising propane are a temperature in the range of 10 to 60° C. and a pressure in the range of 1,000 to 5,000 psig.

12. The method of claim 1 wherein said hydrophobic drug is a Bryostatin.

13. The method of claim 12 wherein said Bryostatin is Bryostatin-1 and derivatives thereof.

14. A therapeutic product comprising of a hydrophobic drug encapsulated in a phospholipid liposome having an average diameter between 0.001 and 200 nanometers made by:
a) providing a solution of a phospholipid material dissolved in a first fluid, said first fluid consisting of a supercritical, critical or near-critical fluid with or without polar cosolvent;
b) forming a solution of a hydrophobic drug in a second fluid, said second fluid is an alcohol;
c) mixing phospholipid-enriched solution in first fluid with a solution of a hydrophobic drug in in an alcoholic solution;
d) depressurizing said phospholipid material and hydrophobic drug solution, and as said phospholipid material and hydrophobic drug exits one or more orifices in the presence of a low solubility fluid, said low solubility fluid having low volatility and said phospholipid material and hydrophobic drug in concentrations which exceed said solubility of said phospholipid material and hydrophobic drug in said low solubility fluid, said phospholipid material and hydrophobic drug forming phospholipid nanosomes having an average diameter between 0.001 and 200 nanometers and said first fluid removed during depressurization;
e) freezing and thawing said phospholipid material and hydrophobic drug solution in a microgravity environment and forming phospholipid nanosomes having an average diameter between 0.001 and 200 nanometers; and
f) wherein at least one step is performed in a said microgravity environment.

15. The product of claim 14 wherein said nanosomes have an average diameter of between 0.001 to 20.000 nanometer.

16. The product of claim 14 wherein said hydrophobic drug is an alpha-secretase modulator.

17. The product of claim 16 wherein said alpha-secretase modulator is Bryostatin-1 and Bryostatin-1 derivatives thereof.

18. A method of making phospholipid nanosomes, comprising the steps of:
a) providing a solution of a phospholipid material drug dissolved in a first fluid, said first fluid consisting of a supercritical, critical or near-critical fluid;
b) forming a solution of a hydrophobic drug in a second fluid, said second fluid is an alcohol;
c) mixing phospholipid-enriched solution in first fluid with a solution of a hydrophobic drug in in an alcoholic solution; and
d) depressurizing said phospholipid material and hydrophobic drug solution to ambient pressure in a microgravity environment wherein said phospholipid material and hydrophobic drug exits one or more orifices in the presence of a low solubility fluid, said low solubility fluid having low volatility and said phospholipid material and hydrophobic drug in concentrations which exceed said solubility of said phospholipid material and hydrophobic drug in said low solubility fluid, said phospholipid material and hydrophobic drug forming phospholipid nanosomes having an average diameter between 0.001 and 200.000 nanometer.

19. The method of claim 18 wherein said wherein second solution for dissolving hydrophobic drug consists of a supercritical, critical or near-critical fluid with or without polar cosolvent.

20. The method of claim 18 wherein said low solubility fluid further includes a hydrophilic drug.
